(19) United States
(12) Patent Application Publication
(10) Pub. No.: US 2012/0238994 A1
(43) Pub. Date: Sep. 20, 2012

(54) TWO-PIECE INJECTABLE DRUG DELIVERY DEVICE WITH HEAT-CURED SEAL

(76) Inventors: Martin Nazzaro, Quincy, MA (US); Josh York, Ipswich, MA (US); Paul Ashton, Newton, MA (US)

(21) Appl. No.: 13/333,743

(22) Filed: Dec. 21, 2011

Related U.S. Application Data
(60) Provisional application No. 61/425,925, filed on Dec. 22, 2010.

(51) Int. Cl. A61M 5/00; B23P 17/04

(52) U.S. Cl. 604/500; 604/57; 29/428

(57) ABSTRACT

The invention provides a drug delivery device for latanoprost or latanoprost acid. The device has a core of latanoprost or latanoprost acid which is surrounded by an internal and external sheath. The external sheath has a first cap that is permeable to latanoprost or latanoprost acid. The first cap may comprise polyvinyl alcohol (PVA), and the PVA may be heat cured. In certain aspects, there are one or more additional caps on the ends of the sheaths formed from one or more polymers. In certain aspects, one or more portions of the drug delivery device is substantially impermeable to latanoprost or latanoprost acid. In certain aspects, the latanoprost or latanoprost acid elutes through the first cap into a biological environment. The invention further provides methods for manufacturing the drug delivery device.

### RELATED APPLICATIONS

This application claims the benefit of U.S. Provisional Application No. 61/425,925, filed Dec. 22, 2010, the contents of which are incorporated by reference in their entirety.

### BACKGROUND

1. Field of the Invention

The present invention relates to injectable drug delivery devices, and processes useful for making and administering such devices.

2. Background of the Invention

The human eye is a highly evolved and complex sensory organ. Damages to any of its essential structures can result in impairment of vision. Treatments of various eye conditions and diseases often consist of applying doses of appropriate medications in aqueous suspension solutions or ointments. While such treatments are satisfactory for conditions where only one or a few applications of the medicinal agents are necessary, certain conditions require more frequent doses, and such treatments are inconvenient to patients. In contrast, injectable drug delivery devices can provide sustained release of a drug in appropriate doses over a period of time without requiring the patient to keep track of when a dosage is to be administered. This is particularly advantageous when a drug must be administered over a period of several weeks or months. Additionally, when the drug must be administered by injection, as in intraocular injections, it is advantageous for the patient to be able to obtain all required doses from a single injection of a drug delivery device instead of having to endure an injection for each dose.

Direct injection into a sensitive and delicate structure like the eye has certain challenges and attendant difficulties. There are a number of procedures and devices that have been developed for the controlled injection of an implant into a tissue, such as an eye. However, improved procedures and devices would be beneficial.

### SUMMARY OF THE INVENTION

In one aspect, the invention provides a drug delivery device comprising a core comprising latanoprost or latanoprost acid, a first sheath at least partially surrounding the core, the first sheath having a first and second end, a second sheath disposed about an exterior surface of the first sheath, the second sheath having a first and second end, and a first cap covering the second end of the second sheath, wherein the first cap is permeable to latanoprost or latanoprost acid in the core, and wherein the first cap is adjacent to the second end of the first sheath. Latanoprost acid is also known as (5Z,9α,11α,15R)-9,11,15-trihydroxy-17-phenyl-18,19,20-trinor-prost-5-en-1-oic acid while latanoprost is also known as isopropyl (5Z,9α,11α,15R)-9,11,15-trihydroxy-17-phenyl-18,19,20-trinor-prost-5-en-1-oate.

In certain embodiments, the device further comprises a second cap covering the first end of the first sheath. In certain such embodiments, the first sheath and the second cap are integrally formed as a single unitary structure. Preferably, the second cap is substantially impermeable to latanoprost or latanoprost acid in the core. The second cap may be formed from one or more polymers, e.g., one or more biodegradable polymers, such as poly(lactic-co-glycolic acid) (PLGA). In preferred such embodiments, the PLGA comprises lactic acid (L) and glycolic acid (G) monomers in a ratio of about 95% L and 5% G.

In certain preferred embodiments, at least one of the first or second sheaths is substantially impermeable to latanoprost or latanoprost acid in the core. Either or both of the first and second sheaths may be formed from one or more polymers, e.g., one or more biodegradable polymers, such as poly(lactic-co-glycolic acid) (PLGA). In preferred such embodiments, the PLGA comprises lactic acid (L) and glycolic acid (G) monomers in a ratio of about 95% L and 5% G.

In certain preferred embodiments, the first end of the second sheath is covered by a seal, e.g., an impermeable seal, such as a silicone seal. The impermeable seal may maintain the first and second sheaths in a fixed spatial relationship relative to each other.

In certain embodiments, when the device is placed in a biological environment, latanoprost or latanoprost acid in the core elutes, e.g., is released from the device, according to a substantially zero-order release profile, through the first cap into the biological environment, e.g., substantially exclusively through the first cap into the biological environment. In preferred embodiments, the first and second sheaths (and optionally the first cap) do not substantially biodegrade in a biological environment prior to release of at least 90% of latanoprost or latanoprost acid in the core.

In certain embodiments, the second sheath substantially surrounds the first sheath, e.g., the first sheath has a longitudinal dimension slightly smaller than a longitudinal dimension of the second sheath. In certain embodiments, the first and second sheaths are cylindrical in shape. The first sheath is preferably dimensionally stable and retains its shape in the absence of the core and the second sheath. The second sheath is preferably dimensionally stable and retains its shape in the absence of the core and the first sheath. The first sheath may be frictionally engaged with the second sheath.

The first cap may be formed from one or more polymers, e.g., one or more biodegradable polymers. In certain embodiments, the first cap comprises poly(vinyl alcohol) (PVA), preferably heat-cured PVA.

In another aspect, the invention provides a method for manufacturing an injectable drug delivery device comprising providing a first sheath having a first and second end, placing latanoprost or latanoprost acid into an interior region of the first sheath to form a drug core at least partially surrounded by the first sheath, providing a second sheath having a first and second end, and inserting the first sheath into an interior region of the second sheath such that the second sheath at least partially surrounds the first sheath.

In certain preferred embodiments, the method further comprises sealing the second end of the second sheath with a first cap permeable to latanoprost or latanoprost acid in the drug core, e.g., before or after inserting the first sheath into the second sheath, preferably before. In certain such embodiments, the first cap is formed from one or more polymers, e.g., one or more biodegradable polymers. In certain embodiments, the method preferably further comprises heat-curing the first cap prior to inserting the first sheath into the second sheath, particularly in preferred embodiments in which the first cap comprises poly(vinyl alcohol) (PVA).

In certain embodiments, the method further comprises sealing the first end of the first sheath with a second cap, e.g., before or after placing the latanoprost or latanoprost acid into the interior region of the first sheath. In alternative embodiments, the first sheath is closed at the first end by a second cap formed integrally with the first sheath. Preferably, the second cap is substantially impermeable to latanoprost or latanoprost acid in the core. The second cap may be formed from one or more polymers, e.g., one or more biodegradable polymers, such as poly(lactic-co-glycolic acid) (PLGA). In preferred such embodiments, the PLGA comprises lactic acid (L) and glycolic acid (G) monomers in a ratio of about 95% L and 5% G.

In certain embodiments, when the device is placed in a biological environment, latanoprost or latanoprost acid in the core elutes, e.g., is released from the device, according to a substantially zero-order release profile, through the first cap into the biological environment, e.g., substantially exclusively through the first cap into the biological environment. In preferred embodiments, the first and second sheaths (and optionally either or both of the first and second caps) do not substantially biodegrade in a biological environment prior to release of at least 90% of the latanoprost or latanoprost acid in the core.

In certain preferred embodiments, the method further comprises forming a seal, e.g., an impermeable seal, such as a silicone seal, over the first end of the second sheath. The impermeable seal may maintain the first and second sheaths in a fixed spatial relationship relative to each other.

In certain preferred embodiments, at least one of the first or second sheaths is substantially impermeable to latanoprost or latanoprost acid in the core. Either or both of the first and second sheaths may be formed from one or more polymers, e.g., one or more biodegradable polymers, such as poly(lactic-co-glycolic acid) (PLGA). In preferred such embodiments, the PLGA comprises lactic acid (L) and glycolic acid (G) monomers in a ratio of about 95% L and 5% G.

In certain embodiments, the second sheath substantially surrounds the first sheath after insertion, e.g., the first sheath has a longitudinal dimension slightly smaller than a longitudinal dimension of the second sheath. In certain embodiments, the first and second sheaths are cylindrical in shape. The first sheath is preferably dimensionally stable and retains its shape in the absence of the drug core and the second sheath. The second sheath is preferably dimensionally stable and retains its shape in the absence of the drug core and the first sheath. The first sheath may frictionally engage the second sheath during insertion.

In another aspect, the invention provides a drug delivery device comprising an inner casing comprising a) an inner wall defining a central cavity having a first and second end, an outer casing comprising b) an outer wall disposed about an exterior surface of the inner wall and c) a first outer cap sealing the outer casing adjacent to the second end, and a drug core comprising latanoprost or latanoprost acid disposed in the central cavity, wherein the first outer cap is permeable to latanoprost or latanoprost acid disposed in the central cavity.

In certain embodiments, the inner wall and the outer wall are substantially coextensive. In certain preferred embodiments, the inner wall and the outer wall each have longitudinal dimensions independent of each other and of the drug core. The inner casing is preferably dimensionally stable and retains its shape in the absence of the drug core and the outer casing. The outer casing is preferably dimensionally stable and retains its shape in the absence of the drug core and the inner casing. The inner casing and the outer casing may be slidably engaged.

In certain preferred embodiments, at least one of the inner wall and the outer wall is substantially impermeable to latanoprost or latanoprost acid disposed in the central cavity. Either or both of the inner casing and outer casing may be formed from one or more polymers, e.g., in whole or in part from one or more biodegradable polymers, such as poly(lactic-co-glycolic acid) (PLGA). Preferably at least the inner/outer wall(s) is/are formed from a biodegradable polymer, such as PLGA. In preferred such embodiments, the PLGA comprises lactic acid (L) and glycolic acid (G) monomers in a ratio of about 95% L and 5% G.

In certain embodiments, the device further comprises either i) a second outer cap, e.g., comprising silicone, adjacent to the first end, that contacts the outer wall and maintains the inner wall and the outer wall in fixed positions relative to each other, or ii) an inner cap sealing the first end of the inner casing, or both. Preferably, either or both of the second outer cap and inner cap are substantially impermeable to latanoprost or latanoprost acid disposed in the central cavity. The inner cap may be formed from one or more polymers, e.g., one or more biodegradable polymers, such as poly(lactic-co-glycolic acid) (PLGA). In preferred such embodiments, the PLGA comprises lactic acid (L) and glycolic acid (G) monomers in a ratio of about 95% L and 5% G.

The first outer cap, which preferably abuts the second end of the inner casing, may be formed from one or more polymers, e.g., one or more biodegradable polymers. In certain embodiments, the first outer cap comprises poly(vinyl alcohol) (PVA), preferably heat-cured PVA.

In certain embodiments, when the device is placed in a biological environment, latanoprost or latanoprost acid disposed in the drug core elute, e.g., are released from the device, according to a substantially zero-order release profile, through the first outer cap into the biological environment, e.g., substantially exclusively through the first outer cap into the biological environment. In preferred embodiments, the inner casing and outer casing (and optionally either or both of the first outer cap and inner cap) do not substantially biodegrade in a biological environment prior to release of at least 90% of the latanoprost or latanoprost acid in the drug core.

In another aspect, the invention provides a method for manufacturing a drug delivery device comprising providing an inner casing comprising a) an inner wall defining a central cavity having a first and second end, placing latanoprost or latanoprost acid into the central cavity of the inner casing to form a drug core at least partially surrounded by the inner casing, and inserting the inner casing into an outer casing comprising b) an outer wall disposed about an exterior surface of the inner wall and c) a first outer cap sealing the outer casing adjacent to the second end, wherein the first outer cap is permeable to latanoprost or latanoprost acid in the drug core.

The first outer cap, which preferably abuts the second end of the inner casing, may be formed from one or more polymers, e.g., one or more biodegradable polymers. In certain embodiments, the first outer cap is heat-cured, particularly in preferred embodiments in which the first cap comprises poly(vinyl alcohol) (PVA).

In certain embodiments, the method further comprises sealing the first open end of the inner casing with an inner cap, e.g., before or after placing the latanoprost or latanoprost acid into the central cavity of the inner casing. In alternative embodiments, the inner casing is sealed at the first open end by an inner cap formed integrally with the inner casing. Preferably, the inner cap is substantially impermeable to latanoprost or latanoprost acid in the drug core. The inner cap may be formed from one or more polymers, e.g., one or more biodegradable polymers, such as poly(lactic-co-glycolic acid) (PLGA). In preferred such embodiments, the PLGA comprises lactic acid (L) and glycolic acid (G) monomers in a ratio of about 95% L and 5% G.

In certain preferred embodiments, the method further comprises forming a second outer cap, e.g., comprising silicone, adjacent to the first open end that contacts the outer wall. The second outer cap may maintain the inner wall and the outer wall in fixed positions relative to each other. The second outer cap is preferably substantially impermeable to latanoprost or latanoprost acid in the drug core.

In certain preferred embodiments, at least one of the inner wall and the outer wall is substantially impermeable to latanoprost or latanoprost acid in the drug core. Either or both of the inner casing and outer casing may be formed from one or more polymers, e.g., one or more biodegradable polymers, such as poly(lactic-co-glycolic acid) (PLGA). Preferably at least the inner/outer wall(s) is/are formed from a biodegradable polymer, such as PLGA. In preferred such embodiments, the PLGA comprises lactic acid (L) and glycolic acid (G) monomers in a ratio of about 95% L and 5% G.

In certain embodiments, when the device is placed in a biological environment, latanoprost or latanoprost acid in the drug core elutes, e.g., is released from the device, according to a substantially zero-order release profile, through the first outer cap into the biological environment, e.g., substantially exclusively through the first outer cap into the biological environment. In preferred embodiments, the inner casing and outer casing (and optionally either or both of the first outer cap and inner cap) do not substantially biodegrade in a biological environment prior to release of at least 90% of latanoprost or latanoprost acid in the drug core.

In certain embodiments, the inner wall and the outer wall are substantially coextensive. In certain embodiments, the inner casing has a longitudinal dimension slightly smaller than a longitudinal dimension of the outer casing. In certain embodiments, the inner and outer casings are cylindrical in shape. The inner casing is preferably dimensionally stable and retains its shape in the absence of the drug core and the outer casing. The outer casing is preferably dimensionally stable and retains its shape in the absence of the drug core and the inner casing.

In another aspect, the invention provides a drug delivery device comprising an inner casing comprising an inner tube having a first and second end, an outer casing slidably engaged with the inner casing, the outer casing comprising an outer tube disposed about the inner tube and a first outer cap sealing an end of the outer casing adjacent to the second end, and a drug core comprising latanoprost or latanoprost acid disposed in the inner tube, wherein the first outer cap is permeable to latanoprost or latanoprost acid disposed in the central cavity.

In certain embodiments, the device further comprises either i) a second outer cap, e.g., comprising silicone, adjacent to the first end of the inner tube, that contacts the outer tube and maintains the inner tube and the outer tube in fixed positions relative to each other, or ii) an inner cap sealing the first end of the inner tube, or both. Preferably, either or both of the second outer cap and inner cap are substantially impermeable to latanoprost or latanoprost acid disposed in the central cavity. The inner cap may be formed from one or more polymers, e.g., one or more biodegradable polymers, such as poly(lactic-co-glycolic acid) (PLGA). In preferred such embodiments, the PLGA comprises lactic acid (L) and glycolic acid (G) monomers in a ratio of about 95% L and 5% G.

In certain preferred embodiments, at least one of the inner tube and the outer tube is substantially impermeable to latanoprost or latanoprost acid disposed in the central cavity. Either or both of the inner casing and outer casing may be formed from one or more polymers, e.g., one or more biodegradable polymers, such as poly(lactic-co-glycolic acid) (PLGA). Preferably at least the inner/outer tube(s) is/are formed from a biodegradable polymer, such as PLGA. In preferred such embodiments, the PLGA comprises lactic acid (L) and glycolic acid (G) monomers in a ratio of about 95% L and 5% G.

In certain embodiments, when the device is placed in a biological environment, latanoprost or latanoprost acid disposed in the drug core elute, e.g., are released from the device, according to a substantially zero-order release profile, through the first outer cap into the biological environment, e.g., substantially exclusively through the first outer cap into the biological environment. In preferred embodiments, the inner casing and outer casing (and optionally either or both of the first outer cap and inner cap) do not substantially biodegrade in a biological environment prior to release of at least 90% of latanoprost or latanoprost acid in the drug core.

The first outer cap, which preferably abuts the second end of the inner casing, may be formed from one or more polymers, e.g., one or more biodegradable polymers. In certain embodiments, the first outer cap comprises poly(vinyl alcohol) (PVA), preferably heat-cured PVA.

In certain embodiments, the inner tube and the outer tube are substantially coextensive. The inner casing is preferably dimensionally stable and retains its shape in the absence of the drug core and the outer casing. The outer casing is preferably dimensionally stable and retains its shape in the absence of the drug core and the inner casing.

In another aspect, the invention provides a method for manufacturing a drug delivery device comprising providing an inner casing comprising an inner tube having a first and second end, placing latanoprost or latanoprost acid into the inner tube to form a drug core at least partially surrounded by the inner casing, and slidably engaging an outer casing with the inner casing, the outer casing comprising an outer tube disposed about the inner tube and a first outer cap sealing an end of the outer casing adjacent to the second end, wherein the first outer cap is permeable to latanoprost or latanoprost acid in the drug core.

The first outer cap, which preferably abuts the second end of the inner casing, may be formed from one or more polymers, e.g., one or more biodegradable polymers. In certain embodiments, the first outer cap is heat-cured, particularly in preferred embodiments in which the first cap comprises poly(vinyl alcohol) (PVA). In certain preferred embodiments, the method further comprises heat-curing the first outer cap prior to engaging the first casing and the second casing.

In certain embodiments, the method further comprises sealing the first end of the inner casing with an inner cap, e.g., before or after placing the latanoprost or latanoprost acid into the inner tube. In alternative embodiments, the inner casing is sealed at the first end by an inner cap formed integrally with the inner casing. Preferably, the inner cap is substantially impermeable to latanoprost or latanoprost acid in the drug core. The inner cap may be formed from one or more polymers, e.g., one or more biodegradable polymers, such as poly(lactic-co-glycolic acid) (PLGA). In preferred such embodiments, the PLGA comprises lactic acid (L) and glycolic acid (G) monomers in a ratio of about 95% L and 5% G.

In certain preferred embodiments, the method further comprises forming a second outer cap, e.g., comprising silicone, adjacent to the first end that contacts the outer tube and maintains the inner tube and the outer tube in fixed positions relative to each other. The second outer cap is preferably substantially impermeable to latanoprost or latanoprost acid in the drug core.

In certain preferred embodiments, at least one of the inner tube and the outer tube is substantially impermeable to latanoprost or latanoprost acid in the drug core. Either or both of the inner casing and outer casing may be formed from one or more polymers, e.g., one or more biodegradable polymers, such as poly(lactic-co-glycolic acid) (PLGA). Preferably at least the inner/outer tube(s) is/are formed from a biodegradable polymer, such as PLGA. In preferred such embodiments, the PLGA comprises lactic acid (L) and glycolic acid (G) monomers in a ratio of about 95% L and 5% G.

In certain embodiments, when the device is placed in a biological environment, latanoprost or latanoprost acid in the drug core elutes, e.g., is released from the device, according to a substantially zero-order release profile, through the first outer cap into the biological environment, e.g., substantially exclusively through the first outer cap into the biological environment. In preferred embodiments, the inner casing and outer casing (and optionally either or both of the first outer cap and inner cap) do not substantially biodegrade in a biological environment prior to release of at least 90% of the latanoprost or latanoprost acid in the drug core.

In certain embodiments, the inner tube and the outer tube are substantially coextensive. In certain embodiments, the inner casing has a longitudinal dimension slightly smaller than a longitudinal dimension of the outer casing. In certain embodiments, the inner and outer casings are cylindrical in shape. The inner casing is preferably dimensionally stable and retains its shape in the absence of the drug core and the outer casing. The outer casing is preferably dimensionally stable and retains its shape in the absence of the drug core and the inner casing.

The invention further provides drug delivery devices prepared by any of the methods disclosed herein, preferably shaped and sized for injection, e.g., into the eye of a patient.

The present invention further provides methods for delivering a drug to an animal, comprising implanting into the animal (e.g., inserting, preferably injecting, e.g., into an eye of the animal) a drug delivery device as described herein, whereby latanoprost or latanoprost acid diffuses out of the drug delivery device into the animal after implantation. In certain embodiments, the device provides an effective amount of latanoprost or latanoprost acid for at least about a week, at least about a month, or even at least about six months. In certain embodiments, the animal is a mammal, preferably a primate, such as a human.

### BRIEF DESCRIPTION OF THE DRAWINGS

The invention of the present application will now be described in more detail with reference to the accompanying drawings, wherein like reference numerals designate identical or corresponding elements:

FIG. 1 shows components of a drug delivery device prior to assembly;

FIG. 2A shows inner and outer shells of a drug delivery device in the process of assembly;

FIG. 2B shows a longitudinal section of an assembled inner piece of a drug delivery device;

FIG. 3 shows an exterior view of an assembled drug delivery device;

FIG. 4A shows a first illustrative longitudinal section of an assembled drug delivery device;

FIG. 4B shows a second illustrative longitudinal section of an assembled drug delivery device; and

FIG. 4C shows a cross-section of an assembled drug delivery device.

### DETAILED DESCRIPTION

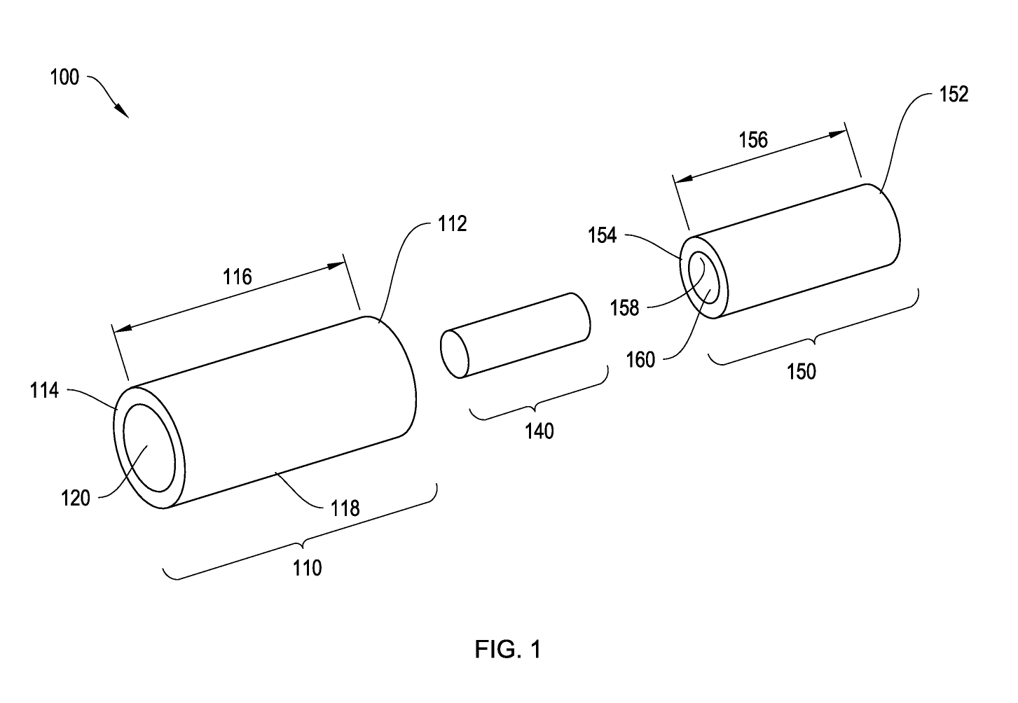

To provide an overall understanding of the invention, certain illustrative embodiments will now be described, including systems and methods for two-piece injectable drug delivery devices having a telescoping assembly. It will be understood that the systems and methods described herein may be usefully applied to a number of different devices, such as devices with various cross-sectional geometries or devices with two or more concentrically aligned or non-concentrically aligned cores of latanoprost or latanoprost acid. It will further be appreciated that various combinations of latanoprost or latanoprost acid and outer layers described herein, or outer layers not specifically mentioned herein, are within the scope of this disclosure and may be usefully employed in an injectable drug delivery device of the present invention. Where an element is not specified as being permeable or impermeable, it will be understood that it may be either permeable or impermeable. All such embodiments are intended to fall within the scope of the invention described herein. FIG. 1 shows components of a drug delivery device prior to assembly. As illustrated in FIG. 1, a device 100 may include an outer shell 110, an inner shell 150, and a drug core 140. Outer shell 110, illustrated with diagonal lines running from upper right to bottom left, may have a first end 112, a second end 114, and an outer wall 118 defining an interior region 120. Inner shell 150, sized to fit within interior region 120 and illustrated with diagonal lines running from upper left to bottom right, may have a first end 152, a second end 154, and an inner wall 158 defining an interior region 160. Inner shell 150 may have a longitudinal dimension 156 that is slightly smaller than a longitudinal dimension 116 of outer shell 110. Drug core 140, illustrated with a dotted pattern, includes latanoprost or latanoprost acid to be delivered by drug delivery device 100 and is sized to fit within interior region 160. In preferred embodiments, at least one of outer shell 110 and inner shell 150 is substantially impermeable to latanoprost or latanoprost acid in drug core 140.

Drug core 140 may be in the form of a solid matrix, a mixture of particulates, a liquid suspension, a paste, or any other suitable form. Drug core 140 may be pre-formed by extrusion, compression, or other means and then optionally sprayed or otherwise coated with a film of material having suitable properties. Alternatively, drug core 140 may be formed in-situ by placing a drug-containing material into interior region 160.

In a preferred embodiment, second end 114 of outer shell 110 is covered by a first cap that is permeable to latanoprost or latanoprost acid in drug core 140. As used herein, the term “cap”, “cover”, or “seal” is intended to mean, but is not limited to, a material covering an opening (e.g., a lid), a plug inserted into an opening, or another flow rate-affecting structure, or an act of forming or putting in place such a material, plug, or structure, e.g., such that no gaps or channels remain through which a fluid, such as water, can pass; rather, any passage of liquid through the covered opening is dependent on the permeability (or not) of the materials covering and defining the opening. The first cap may be formed from one or more polymers that may be biodegradable. In a preferred embodiment, the first cap comprises poly(vinyl alcohol) (PVA). The PVA may be heat-cured to form the first cap. In a preferred embodiment, the heat-cured first cap is formed by applying a PVA solution to second end 114 of outer shell 110, and then heating outer shell 110 (e.g., in an oven or other heating element) at a temperature in the range of 60-120° C., e.g., 80° C., for at least 2 hours, preferably at least 4 hours, e.g., 5 hours. Heat curing temperatures may range between 40° C.-120° C., and heating times may range between 2-24 hours.

In some embodiments, first end 152 of inner shell 150 may be covered by a second cap. The second cap may be substantially impermeable to latanoprost or latanoprost acid in drug core 140. In some embodiments, the second cap may be integrally formed as part of inner shell 150. In other embodiments, the second cap may be formed separately from or attached to first end 152 after inner shell 150 has already been formed.

As used herein, the term “permeable” is intended to mean permeable or substantially permeable to a given substance, e.g., latanoprost or latanoprost acid that the device delivers. As used herein, the term “impermeable” is intended to mean impermeable or substantially impermeable to a given substance, e.g., latanoprost or latanoprost acid that the device delivers. The terms “permeable” and “impermeable” as used herein, when used in opposition to each other, signify differing levels of permeability relative to other materials in the same device. Preferably, permeable materials are at least 10 times more permeable, preferably at least 100 times more permeable, most preferably at least 500 times more permeable, e.g., to latanoprost or latanoprost acid in the core, than an impermeable material in the same device. If a device contains multiple permeable and/or impermeable materials, the comparison is preferably made between the least permeable material in the most permeable path between the drug core 140 and the external environment (e.g., the permeable first cap over second end 114) and the most impermeable material in the second most permeable path between the drug core 140 and the external environment (e.g., inner wall 158 or outer wall 118, whichever is less permeable). Thus, the comparison is made between the elution rate-controlling material disposed over the primary intended elution path and the elution rate-controlling material disposed over the most significant competing elution path.

Generally speaking, suitable biocompatible polymers for use in the subject devices include, but are not limited to, poly(vinyl acetate) (PVAC), poly(caprolactone) (PCL), ethylene vinyl acetate polymer (EVA), poly(ethylene glycol) (PEG), poly(vinyl alcohol) (PVA), poly(lactic acid) (PLA), poly(glycolic acid) (PGA), poly(lactic-co-glycolic acid) (PLGA), polyalkyl cyanoacrylate, polyurethane, nylons, or copolymers thereof. In polymers including lactic acid monomers, the lactic acid may be D-, L- (e.g., poly-L-lactic acid (PLLA)), or any mixture of D- and L-isomers. In preferred embodiments, outer shell 110, inner shell 150, and the second cap, if present, comprise PLGA. PLGA comprises both lactic acid (L) and glycolic acid (G) monomers. The percentage of L may range between 80-97%. The percentage of G may range between 3-20%. In a preferred embodiment, the PLGA comprises L and G monomers in a ratio of about 95% L and 5% G. Exemplary permeable polymers include PVA and PEG; impermeable polymers include nylons, polyurethane, EVA, and polyalkyl cyanoacrylate. Other polymers may be permeable or impermeable depending on the relative characteristics of the polymer and latanoprost or latanoprost acid in the drug core. Suitable biodegradable polymers include, but are not limited to, poly(caprolactone) (PCL), poly(lactic acid) (PLA), poly(glycolic acid) (PGA), poly(lactic-co-glycolic acid) (PLGA), or copolymers thereof.

It will be appreciated that a material may be permeable to latanoprost or latanoprost acid and also substantially control the rate at which the drug diffuses or otherwise passes through the material. Consequently, a permeable membrane may also be a release-rate-limiting or release-rate-controlling membrane, and the permeability of such a membrane may be one of the most significant factors controlling the release rate for a device.

Outer shell 110, inner shell 150, and the second cap, if present, may be formed from one or more polymers that may be biodegradable.

A drug delivery device may be assembled as follows. Latanoprost or latanoprost acid may be placed into interior region 160 of inner shell 150 to form drug core 140. In a preferred embodiment, a latanoprost/silica (e.g., Cab-O-Sil) granulation is injected into interior region 160. In other embodiments, latanoprost- or latanoprost acid-containing mixtures or compositions may be deposited, poured, or otherwise inserted into interior region 160. Inner wall 158 at least partially surrounds drug core 140. In preferred embodiments, drug core 140 lies substantially or completely inside inner shell 150. Inner shell 150 with drug core 140 inside may then be inserted into interior region 120 of outer shell 110 such that outer shell 110 at least partially surrounds inner shell 150. In a preferred embodiment, outer shell 110 substantially surrounds inner shell 150. As used herein, the phrase “substantially surrounds” indicates at least 95% overlap. Assembly is facilitated by outer shell 110, and preferably also inner shell 150, being dimensionally stable, e.g., having the structural ability to accept another element without changing shape, as well as to retain their shape in the absence of the other elements (e.g., capable of supporting their own weight). In addition, having outer shell 110, and preferably also inner shell 150, retain their own structural integrity so that the surface area for diffusion does not significantly change simplifies manufacture of the entire device and enables the device to better deliver latanoprost or latanoprost acid.

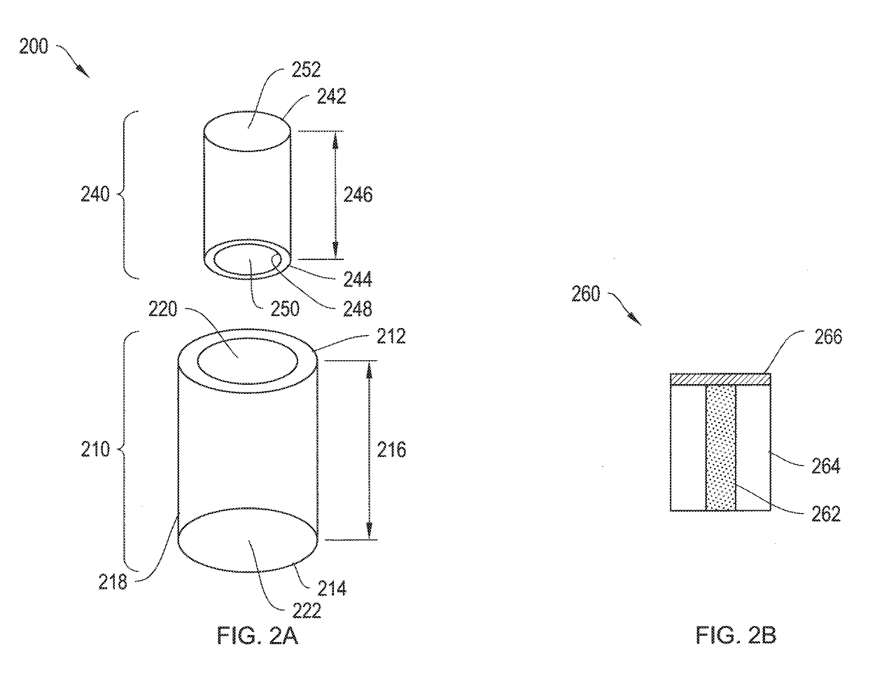

FIG. 2A shows inner and outer shells of a drug delivery device in the process of assembly. Drug delivery device 200 includes an inner shell 240 and an outer shell 210. Inner shell 240, illustrated with diagonal lines running from upper left to bottom right, may have a first end 242 and a second end 244, and an inner wall 248 defining an interior region 250. In some embodiments, inner shell 240 may also have an inner cap 252, illustrated with vertical lines, covering first end 242. Inner wall 248 and inner cap 252 may form a sheath around a drug core that inserted into or formed inside interior region 250. Inner cap 252 may be substantially impermeable to latanoprost or latanoprost acid in the drug core.

FIG. 2B shows a longitudinal section of an assembled inner piece of a drug delivery device, such as inner shell 240 of FIG. 2A. Longitudinal section 260 of FIG. 2B illustrates a profile of a drug core 262, an inner shell 264, and an inner cap 266. Drug core 262 includes latanoprost or latanoprost acid to be delivered by drug delivery device 200.

Longitudinal section 400 includes a profile of a drug core 402, an inner shell 404, an outer shell 408, and a first outer cap 410 covering one end of outer shell 408.

Returning to FIG. 2A, inner cap 252 may be substantially impermeable to latanoprost or latanoprost acid in the drug core surrounded by inner wall 248. In some embodiments, inner cap 252 may be integrally formed as part of inner shell 240. In other embodiments, inner cap 252 may be attached to first end 242 after inner shell 240 has already been formed. In embodiments where inner cap 252 is formed separately from the rest of inner shell 240, inner cap 252 may be coupled either before or after a drug core has been inserted into or formed inside interior region 250.

After a drug core is inserted into or formed inside interior region 250, inner shell 240, with the drug core inside, may be inserted, second end 244 first, into an interior region 220 of outer shell 210 through first end 212. Outer shell 210, illustrated with diagonal lines running from upper right to bottom left, may also have a second end 214 and an outer wall 218 defining interior region 220. In a preferred embodiment, second end 214 of outer shell 210 may be sealed by a first cap 222, illustrated with horizontal lines, that is permeable to latanoprost or latanoprost acid in the drug core. First cap 222 may be formed from one or more polymers that may be biodegradable. In a preferred embodiment, first cap 222 comprises poly(vinyl alcohol) (PVA). The PVA may be heat-cured to form first cap 222. In a preferred embodiment, heat-cured first cap 222 is formed by applying a PVA solution to second end 214 of outer shell 210, and then heating outer shell 210 (e.g., in an oven or other heating element) at a temperature in the range of 60-120° C., e.g., 80° C., for at least 2 hours, preferably at least 4 hours, e.g., 5 hours.

First cap 222 may be formed before inner shell 240 is inserted into outer shell 210, particularly in embodiments where heat curing of first cap 222 is desired and latanoprost or latanoprost acid in the drug core is heat-sensitive. The devices and methods of the invention allow first cap 222 to benefit from the improved structural characteristics that result from heat-curing without the need to subject components of inner shell 240, particularly the drug core, to the heat-curing conditions. This can be particularly advantageous when the drug core contains unstable or temperature-sensitive components, such as biologic agents (proteins, antibodies, etc.) or other heat-labile agents.

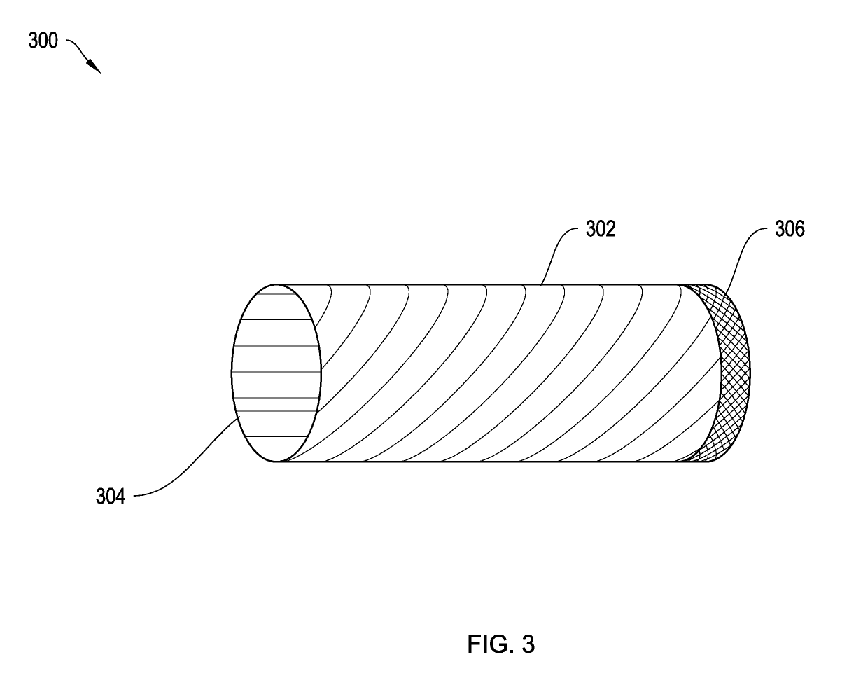

Inner shell 240 may be inserted into outer shell 210 such that outer shell 210 is disposed about the exterior surface of inner shell 240. In a preferred embodiment, outer shell 210 substantially surrounds inner shell 240. In some embodiments, as inner shell 240 is inserted into outer shell 210, inner shell 240 and outer shell 210 may be frictionally or slidably engaged. In some embodiments, one or both of inner shell 240 and outer shell 210 may have one or more longitudinal grooves to allow air to escape as inner shell 240 is inserted into outer shell 210. In some embodiments, the diameter of inner shell 240 may be smaller than the diameter of outer shell 210 such that longitudinal contact occurs around less than the entire interface between inner shell 240 and outer shell 210 during or after insertion Inner shell 240 may have a longitudinal dimension 246 that is slightly smaller than a longitudinal dimension 216 of outer shell 210 such that first cap 222 is adjacent to second end 244 of inner shell 240 when insertion is complete. After inner shell 240 has been inserted into outer shell 210, first end 212 of outer shell 210 may be sealed with an impermeable cap, as discussed below in relation to FIG. 3.

In certain embodiments, a significant factor affecting the release rate of latanoprost or latanoprost acid from a device is the cross-sectional area of interior region 220, which relates to the exposed surface area available for drug diffusion. Thus, in addition to the permeability of first cap 222, the cross-sectional area of interior region 220 is a factor affecting latanoprost or latanoprost acid release rate.

It will be appreciated that other techniques may be employed to preform longitudinally hollow segments useful for making the injectable drug delivery devices described herein. One technique that has been successfully employed is to dip a wire, such as Nitinol, of suitable outer diameter into an uncured polymer or polymer solution. The polymer then may be dried or otherwise cured. The wire may then be withdrawn from the polymer coating to provide a polymer tube into which desired drug formulations may be injected or otherwise inserted.

FIG. 3 shows an exterior view of an assembled drug delivery device. Drug delivery device 300 includes an outer shell 302 surrounding an inner shell and a drug core, as described in greater detail above, and can be assembled using any of the methods and materials described above. Outer shell 302 is sealed at one end with a first outer cap 304 that is permeable to latanoprost or latanoprost acid in the drug core. In a preferred embodiment, first outer cap 304 is formed by heat curing PVA that has been deposited at one end of outer shell 302. In some embodiments, the other end of outer shell 302 is sealed by a second outer cap 306. A function of second outer cap 306 is to retain the inner shell and drug core in outer shell 302, especially in embodiments where the inner shell has no inner cap. Second outer cap 306 may comprise silicone or another material or polymer, preferably one that is substantially impermeable to latanoprost or latanoprost acid in the drug core. In some embodiments, second outer cap 306 maintains the inner shell and the outer shell in fixed positions relative to each other. In a preferred embodiment, second outer cap 306 is formed by depositing silicone adhesive on an end of outer shell 302 and allowing the silicone adhesive to dry, e.g., for at least 72 hours

When device 300 is placed in a biological fluid or environment, latanoprost or latanoprost acid in the drug core elutes through first outer cap 304 into the biological fluid/environment. In a preferred embodiment, when device 300 is placed in a biological fluid or environment, latanoprost or latanoprost acid in the drug core elutes substantially exclusively (e.g., at least 90%, preferably at least 95%, or even at least 99%) through first outer cap 304 into the biological fluid/environment. Placing device 300 into a biological environment may involve implanting, injecting, or inserting the device into an animal or human patient. In some methods of treatment, device 300 may be injected into an eye of a patient, e.g., a patient suffering from primary open angle glaucoma (POAG) or ocular hypertension (OHT). Once inside the patient, latanoprost or latanoprost acid in the drug core may be released from the device (e.g. via diffusion) according to a substantially zero-order release profile.

Device 300 may be particularly suitable for treating ocular conditions such as glaucoma or ocular hypertension. Device 300 may also be particularly suitable for use as an ocular device in treating mammalian organisms, both human and for veterinarian use, and may be surgically implanted within the vitreous of the eye.

Other disease states may also be prevented or treated using the drug delivery device of the present invention. Such disease states are known by those of ordinary skill in the art. For those not skilled in the art, reference may be made to Goodman and Gilman, The Pharmacological Basis of Therapeutics, 8th Ed., Pergamon Press, NY, 1990; and Remington's Pharmaceutical Sciences, 18th Ed., Mack Publishing Co., Easton, Pa., 1990; both of which are incorporated by reference herein.

Different drug delivery devices may be assembled or manufactured for various dosage levels. An exemplary method for manufacturing a high-dose device (e.g., initial release of approximately 4 μg latanoprost/day which slows to approximately 1 μg latanoprost/day after 10 days) is as follows. Inner shell PLGA 95/5 tubes are attached to needle hubs to allow for injection of a granulate into the central cavity of the tubes to form a drug core. Exemplary diameters for an inner shell for a high-dose device are 0.011″+/−0.001″ for the inner diameter and 0.0145″+/−0.001″ for the outer diameter. A granulate (latanoprost/Cab-O-Sil) is produced for injection into the inner shell PLGA 95/5 tubes. The granulate is filled into separate, inner shell PLGA 95/5 tubes and then cut to desired lengths using a Core Cutting Fixture. Outer shell PLGA 95/5 tubes have three drops of 10% PVA applied to one end of the tubes and are oven heated at 80° C. for 5 hours to heat-cure the PVA. Exemplary diameters for an outer shell are 0.016″+/−0.001″ for the inner diameter and 0.018″+/−0.001″ for the outer diameter. Each inner shell is placed into an outer shell containing the heat-cured PVA membrane. Once the inner shell is in place, the other end of the outer shell (i.e., the end without the heat-cured PVA membrane) is treated with one drop of silicone adhesive and is allowed to dry for no less than 72 hours.

An exemplary method for manufacturing a low-dose latanoprost or latanoprost acid device (1 μg/day slowing to 0.2 μg/day after approximately 10 days) is analogous to the method described above for the high-dose device, except that an inner shell with a smaller inner wall diameter (e.g., 0.0061″+/−0.001″) is used, i.e., the inner shell has increased wall thickness. Also, the ratio of Cab-O-Sil to latanoprost for the granulation may be less (e.g., 10% less Cab-O-Sil by weight) than is used for the high-dose devices due to the smaller inner wall diameter of the PLGA 95/5 tubes.

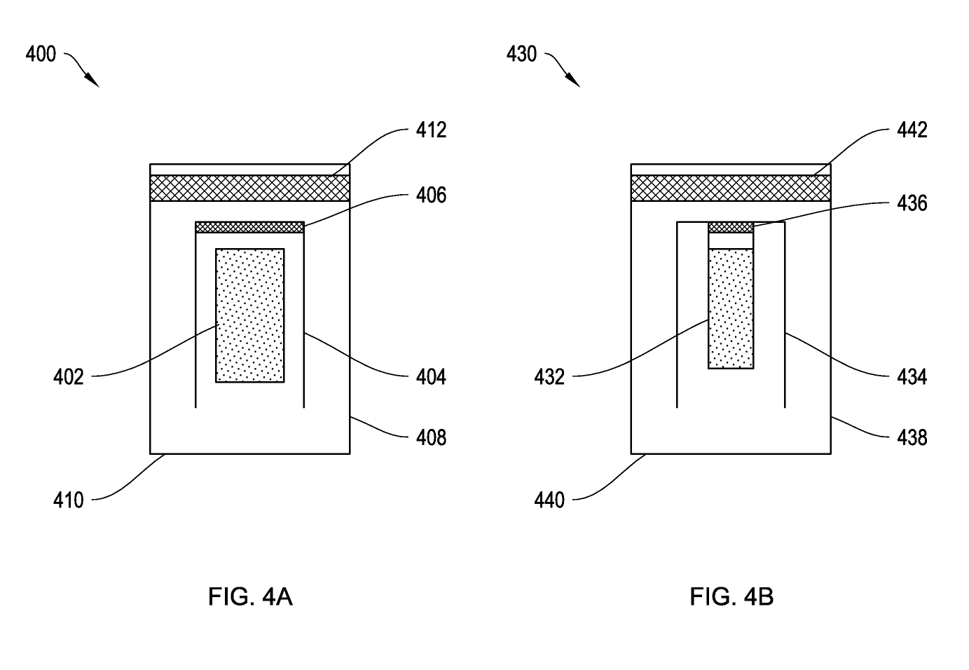

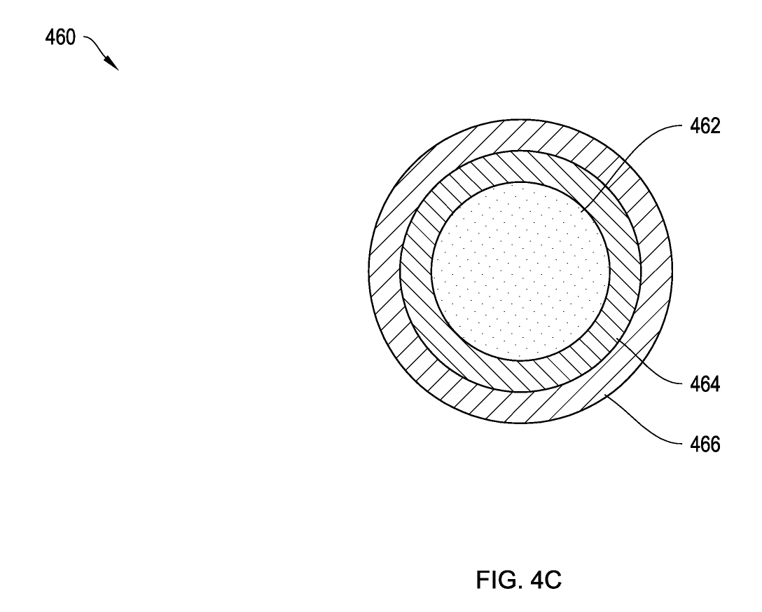

FIG. 4A shows a first illustrative longitudinal section of an assembled drug delivery device, such as device 300 shown in FIG. 3. Longitudinal section 400 includes a profile of a drug core 402, an inner shell 404, an outer shell 408, and a first outer cap 410 covering one end of outer shell 408. In FIG. 4A, there is some space between first outer cap 410 and inner shell 404. However, in some embodiments, first outer cap 410 abuts one end of inner shell 404. In some embodiments, one end of inner shell 404 is covered by an inner cap 406. Inner cap 406, if present, may be impermeable to latanoprost or latanoprost acid in drug core 402. Inner cap 406 may be separately formed from or integrally formed with the walls of inner shell 404. In some embodiments, the end of outer shell 408 that is not covered by first outer cap 410 is covered by a second outer cap 412, which is preferably impermeable to latanoprost or latanoprost acid in drug core 402.

FIG. 4B shows a second illustrative longitudinal section of an assembled drug delivery device, such as device 300 shown in FIG. 3. Longitudinal section 430 includes a profile of a drug core 432, an inner shell 434, an outer shell 438, and a first outer cap 440 covering one end of outer shell 438. FIG. 4B, for the purpose of clarity, shows some space between first outer cap 440 and inner shell 434; however, in preferred embodiments, first outer cap 440 abuts one end of inner shell 434. In some embodiments, one end of inner shell 434, opposite first outer cap 440, is covered by an inner cap 436. Inner cap 436 may be impermeable to latanoprost or latanoprost acid in drug core 432. In the embodiment illustrated in FIG. 4B, inner cap 436 has been formed separately (e.g., attached as a lid or inserted as a plug) from the rest of inner shell 434. In some embodiments, the end of outer shell 438 that is not covered by first outer cap 440 is covered by a second outer cap 432, which is preferably also impermeable to latanoprost or latanoprost acid in drug core 432.

FIG. 4C shows a cross-section of an assembled drug delivery device, such as device 300 shown in FIG. 3. Cross-section 460 shows a drug core 462 surrounded by an inner shell 464, which is in turn surrounded by an outer shell 466. In the embodiment illustrated in FIG. 4C, drug core 462 is in contact with the inner wall of inner shell 464. However, in other embodiments, some space exists between drug core 462 and the inner wall of inner shell 464. Also, in the embodiment illustrated in FIG. 4C, outer shell 466 fits snugly around inner shell 464. However, in other embodiments, the diameter of inner shell 464 may be smaller and hence some space may exist between inner shell 464 and outer shell 466.

A drug delivery device as illustrated in any of the above figures may be shaped and sized for injection (e.g., less than about 4 mm long and less than about 0.5 mm in diameter, e.g., to fit through at least one of a needle having a size from about 30 gauge to about 15 gauge or a cannula having a size from about 30 gauge to about 15 gauge, preferably to fit through a less than 22-gauge cannula). Once injected into a patient, the device may provide an effective amount of latanoprost or latanoprost acid to the patient for an extended period of time (e.g., at least a week, at least a month, or even at least six months). The materials used to make the device may be selected to be substantially stable during the release period of latanoprost or latanoprost acid. The materials may optionally be selected so that, after the drug delivery device has released the latanoprost or latanoprost acid for a predetermined amount of time, the device biodegrades (erodes in situ, i.e., is bioerodible). Biodegradability of the device overcomes the need for retrieval of the device at the end of its lifespan and limits the accumulation of such devices inside the patient. If elements of the device, such as the inner and outer shells (and optionally also the first outer cap) are formed from biodegradable polymers, these elements preferably do not substantially biodegrade (e.g., such that the release rate is affected or the physical integrity or durability of the device is compromised) in a biological environment prior to release of at least 90%, preferably at least 95%, of latanoprost or latanoprost acid in the drug core. The materials may also be selected so that, for the desired life of the delivery device, the materials are stable and do not significantly erode, for example if the inner and outer shells degraded such that, while remaining impermeable, the shells were easily fractured, thereby exposing the drug core directly to the surrounding environment, this would indicate that the shells do substantially biodegrade during the desired life of the device. Optionally, the materials may be chosen to be biodegradable at rates that control, or contribute to control of, the release rate of latanoprost or latanoprost acid. It will be appreciated that other materials, such as additional coatings on some or all of the device may be similarly selected for their biodegradable properties.

While the invention has been described in detail with reference to preferred embodiments thereof, it will be apparent to one skilled in the art that various changes can be made, and equivalents employed, without departing from the scope of the invention. In addition to the embodiments illustrated above, those skilled in the art will understand that any of a number of devices and formulations may be adopted for use with the systems described herein. Thus, the invention set forth in the following claims is to be interpreted in the broadest sense allowable by law. Each of the aforementioned references and published documents is incorporated by reference herein in its entirety.

1. A drug delivery device comprising:
a core comprising latanoprost or latanoprost acid;
a first sheath at least partially surrounding the core, the first sheath having a first and second end;
a second sheath disposed about an exterior surface of the first sheath, the second sheath having a first and second end; and
a first cap covering the second end of the second sheath,
wherein the first cap is permeable to latanoprost or latanoprost acid, and wherein the first cap is adjacent to the second end of the first sheath.

2. The drug delivery device of claim 1, further comprising a second cap covering the first end of the first sheath.

3. The drug delivery device of claim 2, wherein the second cap is substantially impermeable to latanoprost or latanoprost acid in the core.

4. The drug delivery device of claim 2, wherein the second cap is formed from one or more polymers.

5. The drug delivery device of claim 4, wherein the second cap comprises one or more biodegradable polymers.

6. The drug delivery device of claim 5, wherein the second cap comprises poly(lactic-co-glycolic acid) (PLGA).

7. The drug delivery device of claim 2, wherein the first sheath and the second cap are integrally formed as a single unitary structure.

8. The drug delivery device of claim 1, wherein at least one of the first or second sheaths is substantially impermeable to latanoprost or latanoprost acid in the core.

9. The drug delivery device of claim 1, wherein the first end of the second sheath is covered by an impermeable seal.

10. The drug delivery device of claim 9, wherein the impermeable seal comprises silicone.

11. The drug delivery device of claim 1, wherein the second sheath substantially surrounds the first sheath.

12. The drug delivery device of claim 1, wherein the first sheath has a longitudinal dimension slightly smaller than a longitudinal dimension of the second sheath.

13. The drug delivery device of claim 1, wherein the first sheath is frictionally engaged with the second sheath.

14. The drug delivery device of claim 1, wherein the first cap is formed from one or more polymers.

15. The drug delivery device of claim 14, wherein the first cap comprises one or more biodegradable polymers.

16. The drug delivery device of claim 14, wherein said first cap comprises poly(vinyl alcohol) (PVA).

17. The drug delivery device of claim 16, wherein the PVA is heat cured.

18. The drug delivery device of claim 1, wherein, when the device is placed in a biological environment, latanoprost or latanoprost acid elutes through the first cap into the biological environment.

19. The drug delivery device of claim 18, wherein, when the device is placed in the biological environment, latanoprost or latanoprost acid elutes substantially exclusively through the first cap into the biological environment.

20. The drug delivery device of claim 1, wherein at least one of the first and second sheaths is formed from one or more polymers.

21. The drug delivery device of claim 20, wherein at least one of the first and second sheaths comprises one or more biodegradable polymers.

22. The drug delivery device of claim 21, wherein the first and second sheaths comprise poly(lactic-co-glycolic acid) (PLGA).

23. The drug delivery device of claim 6 or 22, wherein said PLGA comprises lactic acid (L) and glycolic acid (G) monomers in a ratio of about 95% L and 5% G.

24. The drug delivery device of claim 1, wherein the first and second sheaths and the first cap do not substantially biodegrade in a biological environment prior to release of at least 90% of latanoprost or latanoprost acid in the core.

25. The drug delivery device of claim 1, wherein, when the device is placed in a biological medium, latanoprost or latanoprost acid are released from the device according to a substantially zero-order release profile.

26. The drug delivery device of claim 1, wherein the first and second sheaths are cylindrical in shape.

27. The drug delivery device of claim 1, wherein the first sheath is dimensionally stable and retains its shape in the absence of the core and the second sheath.

28. The drug delivery device of claim 1, wherein the second sheath is dimensionally stable and retains its shape in the absence of the core and the first sheath.

29. The drug delivery device of claim 1, wherein the device is for implantation, injection, or insertion into a patient.

30. The drug delivery device of claim 1, wherein the device is shaped and sized for injection.

31. A method for manufacturing an injectable drug delivery device comprising:
providing a first sheath having a first and second end;
placing latanoprost or latanoprost acid into an interior region of the first sheath to form a drug core at least partially surrounded by the first sheath;
providing a second sheath having a first and second end; and
inserting the first sheath into an interior region of the second sheath such that the second sheath at least partially surrounds the first sheath.

32. A drug delivery device manufactured by the method of claim 31.

33. A method for delivering a drug to an animal, comprising implanting a drug delivery device according to claim 1 or 32 into the animal, whereby latanoprost or latanoprost acid diffuses out of the drug delivery device into the animal after implantation.

34. A drug delivery device comprising:
an inner casing comprising a) an inner wall defining a central cavity having a first and second end; an outer casing comprising b) an outer wall disposed about an exterior surface of the inner wall and c) a first outer cap sealing the outer casing adjacent to the second end; and
a drug core comprising latanoprost or latanoprost acid disposed in the central cavity,
wherein the first outer cap is permeable to latanoprost or latanoprost acid disposed in the central cavity.

35. A method for manufacturing a drug delivery device comprising:
providing an inner casing comprising a) an inner wall defining a central cavity having a first and second end;
placing latanoprost or latanoprost acid into the central cavity of the inner casing to form a drug core at least partially surrounded by the inner casing; and
inserting the inner casing into an outer casing comprising b) an outer wall disposed about an exterior surface of the inner wall and c) a first outer cap sealing the outer casing adjacent to the second end,
wherein the first outer cap is permeable to latanoprost or latanoprost acid in the drug core.

36. A drug delivery device manufactured by the method of claim 35.

37. A method for delivering latanoprost or latanoprost acid to an animal, comprising implanting a drug delivery device according to claim 34 or 36 into the animal, whereby latanoprost or latanoprost acid diffuses out of the drug delivery device into the animal after implantation.

38. A drug delivery device comprising:
an inner casing comprising an inner tube having a first and second end;
an outer casing slidably engaged with the inner casing, the outer casing comprising an outer tube disposed about the inner tube and a first outer cap sealing an end of the outer casing adjacent to the second end; and
a drug core comprising latanoprost or latanoprost acid disposed in the inner tube,
wherein the first outer cap is permeable to latanoprost or latanoprost acid disposed in the central cavity.

39. A method for manufacturing a drug delivery device comprising:
providing an inner casing comprising an inner tube having a first and second end;
placing at least on drug selected from latanoprost and latanoprost acid into the inner tube to form a drug core at least partially surrounded by the inner casing; and
slidably engaging an outer casing with the inner casing, the outer casing comprising an outer tube disposed about the inner tube and a first outer cap sealing an end of the outer casing adjacent to the second end,
wherein the first outer cap is permeable to latanoprost or latanoprost acid in the drug core.

40. A drug delivery device manufactured by the method of claim 39.

41. A method for delivering a drug to an animal, comprising implanting a drug delivery device according to claim 38 or 40 into the animal, whereby latanoprost or latanoprost acid diffuses out of the drug delivery device into the animal after implantation.

42. A drug delivery device comprising:
an inner casing comprising an inner tube having a first and second end;
an outer casing slidably engaged with the inner casing, the outer casing comprising an outer tube disposed about the inner tube and a first outer cap sealing an end of the outer casing adjacent to the second end; and
a drug core comprising latanoprost or latanoprost acid disposed in the inner tube,
wherein the inner casing and outer casing comprise poly(lactic-co-glycolic acid) (PLGA) and the first outer cap comprises poly(vinyl alcohol) (PVA).
